(19) United States
(12) Patent Application Publication
(10) Pub. No.: US 2012/0185001 A1
(43) Pub. Date: Jul. 19, 2012

(54) Bone Anchors Compatible for Use with Neural Integrity Monitoring Systems and Procedures

(75) Inventors: Jerome Nayet, Saint Genis Pouilly (FR); William Alan Rezach, Atoka, TN (US); Christopher M. Patterson, Olive Branch, MS (US); Boris Voirol, Grandson (CH); Keith E. Miller, Germantown, TN (US)

(73) Assignee: WARSAW ORTHOPEDIC, INC., Warsaw, IN (US)

(21) Appl. No.: 13/007,207

(22) Filed: Jan. 14, 2011

(51) Int. Cl. A61B 17/86

(52) U.S. Cl. 606/301; 606/300

(57) ABSTRACT

A bone anchor compatible for use with a neural integrity monitoring system. The bone anchor includes a bone engaging portion configured for anchoring to bone and has at least one insulated region coated with a bone growth promoting material and at least one conductive region contiguous with the insulated region and having reduced electrical resistance relative to the insulated region.

### BACKGROUND

Surgery for a patient can be painful and traumatic, particularly in the affected area of the patient's body. With regard to spinal fixation systems, a necessary procedure often involves forming a hole in a vertebra of the patient's spine and inserting a bone anchor, such as a bone screw, into the hole. Pedicle screws are advantageous in that they are strong and provide stability, although many types of bone screws are available for use with spinal fixation systems. However, care must be taken to avoid nerve impingement during formation of the holes and/or placement of the bone screws in the vertebral body. Moreover, placement of bone screws is largely done blindly, and even in the hands of experienced surgeons, the incidence of misplaced bone screws resulting in neurological impairment can be quite high despite the use of surgical inspection and imaging techniques.

Other techniques are sometimes used to avoid nerve impingement during surgical procedures including, for example, monitoring of muscle reactions in response to electrical stimulation to locate nerves in or adjacent to the bone tissue during formation of the holes and/or during insertion of the bone screws. Various types of neural integrity monitoring systems are currently available for locating and identifying peripheral motor nerves during spinal surgery. One such system is the NIM-Spine® System marketed by Medtronic, Inc. However, other neural integrity monitoring systems are also in use.

In some instances, the threaded shank of bone screws used in spinal fixation systems are coated with a bone growth promoting material including, for example, calcium phosphate or hydroxyapatite, to increase the purchase strength of the bone screw with the adjacent bone tissue. However, applying a coating to the threaded shank may render the bone screw incompatible for use with neural integrity monitoring systems since such systems require the transfer of an electrical signal between the threaded shank and the adjacent tissue to provide proper neural monitoring and detection of nerve impingement.

Thus, there remains a need for bone anchors that are compatible for use with neural integrity monitoring systems and procedures. The present invention satisfies this need and provides other benefits and advantages in a novel and unobvious manner.

### SUMMARY

The present invention relates generally to bone anchors that are compatible for use with neural integrity monitoring systems and procedures. While the actual nature of the invention covered herein can only be determined with reference to the claims appended hereto, certain forms of the invention that are characteristic of the preferred embodiments disclosed herein are described briefly as follows.

In one form of the present invention, a bone anchor is provided which is compatible for use with a neural integrity monitoring system and which includes a bone engaging portion configured for anchoring to bone and having at least one insulated region coated with a bone growth promoting material and at least one conductive region contiguous with the insulated region and having reduced electrical resistance relative to the insulated region.

In another form of the present invention, a bone anchor is provided which is compatible for use with a neural integrity monitoring system and which includes an implant engaging portion and a bone engaging portion configured for anchoring in bone and including a shank with at least one thread lead, and with the shank and upper and lower flank surfaces of the thread lead coated with a bone growth promoting material to define an insulted region of the bone engaging portion, and an outer thread crest of the thread lead defining a conductive region of the bone engaging portion having reduced electrical resistance relative to the insulated region.

In further form of the present invention, a bone anchor is provided which is compatible for use with a neural integrity monitoring system and which includes a head and a threaded shank formed of a metallic material and extending from the head. The threaded shank includes at least one thread lead, and the threaded shank is entirely coated with a bone growth promoting material except for a non-coated outer thread crest of the thread lead which defines an exposed metallic surface.

It is one object of the present invention to provide bone anchors that are compatible for use with neural integrity monitoring systems and procedures. Further embodiments, forms, features, aspects, benefits, objects, and advantages of the present application shall become apparent from the detailed description and figures provided herewith.

### BRIEF DESCRIPTION OF THE DRAWINGS

FIG. 1 is a schematic diagram of a neural integrity monitoring system.

FIG. 2 is a diagrammatic side view of a boring tool relative to a section of the spinal column for use in association with the neural integrity monitoring system illustrated in FIG. 1.

FIG. 3 is a diagrammatic side view of a bone anchor driver and a bone anchor relative to a section of the spinal column for use in association with the neural integrity monitoring system illustrated in FIG. 1.

FIG. 4 is a posterior view of a spinal stabilization system including a pair of elongate spinal stabilization rods anchored to a section of the spinal column by a plurality of bone anchors.

FIG. 5 is a perspective view of a bone anchor according to one form of the present invention that is compatible for use with neural integrity monitoring systems and procedures.

FIG. 6 is a perspective view of a bone anchor according to another form of the present invention that is compatible for use with neural integrity monitoring systems and procedures.

FIG. 7 is a perspective view of a bone anchor according to another form of the present invention that is compatible for use with neural integrity monitoring systems and procedures.

FIG. 8 is a perspective view of a bone anchor according to another form of the present invention that is compatible for use with neural integrity monitoring systems and procedures.

FIG. 9 is a perspective view of a bone anchor according to another form of the present invention that is compatible for use with neural integrity monitoring systems and procedures.

FIG. 10 is a perspective view of a bone anchor according to another form of the present invention that is compatible for use with neural integrity monitoring systems and procedures.

FIG. 11 is a perspective view of a bone anchor according to another form of the present invention that is compatible for use with neural integrity monitoring systems and procedures.

FIG. 12 is a perspective view of a bone anchor according to another form of the present invention that is compatible for use with neural integrity monitoring systems and procedures.

FIG. 13 is a perspective view of a bone anchor according to another form of the present invention that is compatible for use with neural integrity monitoring systems and procedures.

### DESCRIPTION OF THE ILLUSTRATED EMBODIMENTS

For the purpose of promoting an understanding of the principles of the present invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

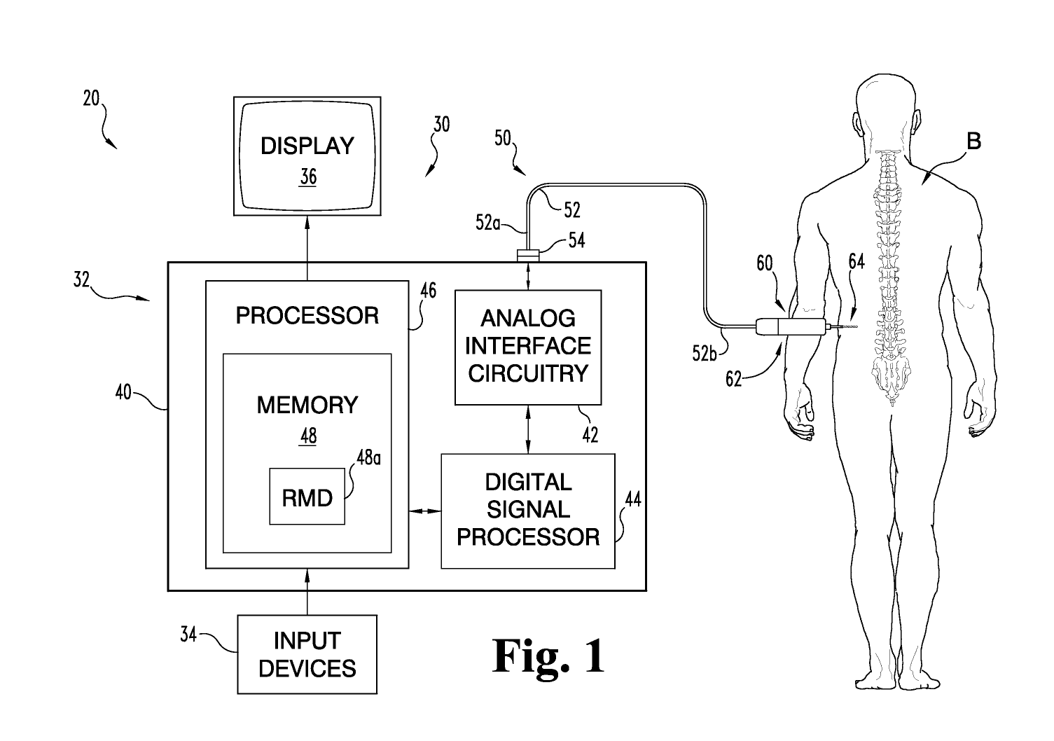

FIG. 1 illustrates a system 20 that may be used, for example, in surgical procedures involving the implantation of spinal stabilization systems to correct a spinal deformity or to stabilize one or more vertebrae of the spinal column B. The system 20 is operable to provide neural integrity or nerve monitoring and to detect impingement or interference between various tools and implants positioned in a vertebral body and neural elements associated with the spinal column B. Upon detection of impingement or interference between the tool and/or implant and the neural element, the system 20 notifies a user of such occurrence so that appropriate remedial or corrective action can be taken during the surgical procedure.

The system 20 generally includes a nerve monitoring system 30, a connection link 50, and a surgical tool 60. The nerve monitoring system 30 includes equipment 32 electrically coupled to the surgical tool 60 via the connection link 50. In a different embodiment, the components of the equipment 32 may be integrated into the surgical tool 60 to provide a stand alone nerve monitoring tool. The surgical tool 60 is configured for operation relative to vertebral bone such as, for example, a vertebral body in the spinal column B of a human patient or subject, as generally represented in FIG. 1. One example of a nerve monitoring system 30 suitable for use in association with the present invention is the NIM-Spine® System marketed by Medtronic, Inc. However, it should be understood that other nerve monitoring systems are also contemplated for use with the present invention.

The equipment 32 generally includes an operator input device 34, an operator display device 36, and various other operator-utilized devices of system 20 that are external to a patient during use. The input devices 34 may include an alphanumeric keyboard and mouse or other suitable pointing/selection input devices. Alternatively or additionally, other input devices can be utilized including, for example, a voice input subsystem or other voice input devices as would occur to those skilled in the art. The operator display device 36 can be of a Cathode Ray Tube (CRT) type, a Liquid Crystal Display (LCD) type, a plasma type, an Organic Light Emitting Diode (OLED) type, or other types of displays as would occur to those skilled in the art. Alternatively or additionally, other output devices may be utilized, such as a printer, one or more loudspeakers, headphones, indicator lights, or other types of output devices as would occur to those skilled in the art. The nerve monitoring system 30 may also include one or more communication interfaces suitable for connection to a computer network, such as a Local Area Network (LAN), a Municipal Area Network (MAN), and/or a Wide Area Network (WAN) such as the Internet, a medical diagnostic device, a therapeutic device, a medical imaging device, a Personal Digital Assistant (PDA) device, a digital still image or video camera, and/or an audio device. The nerve monitoring system 30 can be arranged to show other information or data under control of the operator, the details of which would be apparent to those skilled in the art.

The equipment 32 may also include a processing subsystem 40 for processing signals and data associated with the system 20. The subsystem 40 may generally include analog interface circuitry 42, a Digital Signal Processor (DSP) 44, a data processor 46, and memory 48. The analog interface circuitry 42 can be responsive to control signals from the DSP 44 to provide corresponding analog stimulus signals to the surgical tool 60. At least one of the analog interface circuitry 42 and the DSP 44 may include one or more digital-to-analog converters (DAC) and one or more analog-to-digital converters (ADC) to facilitate operation of the system 20 in a prescribed manner. The processor 46 can be coupled to the DSP 44 to bidirectionally communicate therewith, selectively provide output to display device 36, and/or selectively respond to input from the operator input devices 34.

The DSP 44 and/or the processor 46 can be of a programmable type, a dedicated, hardwired state machine, or a combination thereof. The DSP 44 and the processor 46 perform in accordance with operating logic that can be defined by software programming instructions, firmware, dedicated hardware, a combination of these, or in a different manner as would occur to those skilled in the art. For a programmable form of the DSP 44 or the processor 46, at least a portion of the operating logic can be defined by instructions stored in the memory 48. Programming of the DSP 44 and/or the processor 46 can be of a standard static type, an adaptive type provided by neural networking, expert-assisted learning, fuzzy logic, or a combination thereof.

The memory 48 is illustrated in association with the processor 46. However, the memory 48 can alternatively be separate from or at least partially included in one or both of the DSP 44 and the processor 46. The memory 48 may include a Removable Memory Device (RMD) 48a. Additionally, the memory 48 can be of a solid-state variety, electromagnetic variety, optical variety, or a combination thereof. Furthermore, the memory 48 can be volatile, nonvolatile, or a mixture thereof. The memory 48 can also be at least partially integrated with the circuitry 42, the DSP 44, and/or the processor 46. The RMD 48a can be a floppy disc, cartridge, or tape form of removable electromagnetic recording media, an optical disc such as a CD or DVD type, an electrically reprogrammable solid-state type of nonvolatile memory, and/or other types of memory as would occur to those skilled in the art. In other embodiments, the RMD 48a need not be included in the nerve monitoring system 30.

The circuitry 42, the DSP 44, and the processor 46 can be comprised of one or more components of any type suitable to operate as described herein. It should be appreciated that all or any portion of the circuitry 42, the DSP 44, and the processor 46 can be integrated together in a common device, and/or provided as multiple processing units. For a multiple processing unit form of the DSP 44 or the processor 46, distributed, pipelined, and/or parallel processing can be utilized as appropriate. In one embodiment, the circuitry 42 is provided as one or more components coupled to a dedicated integrated circuit form of the DSP 44, the processor 46 is provided in the form of one or more general purpose central processing units that interface with the DSP 44 over a standard bus connection, and the memory 48 includes dedicated memory circuitry integrated within the DSP 44 and the processor 46, and one or more external memory components including a removable disk form of the RMD 48a. The circuitry 42, the DSP 44, and/or the processor 46 can include one or more signal filters, limiters, oscillators, format converters (such as DACs or ADCs), power supplies, or other signal operators or conditioners as appropriate to operate the system 20 in a prescribed manner.

In one embodiment, the connection link 50 includes an electrical link 52 in the form of a flexible cable having a proximal end 52a and an opposite distal end 52b. A connector 54 is electrically coupled to the equipment 32 of the nerve monitoring system 30. The link 52 extends from the connector 54 at the proximal end 52a to the distal end 52b, with the surgical tool 60 electrically connected to the distal end 52b of the electrical link 52. The connection link 50 may include forms in addition to or alternative to the link 52, including one or more wires, cords, wireless links, infrared components, bluetooth, or other communication links. Furthermore, it should be appreciated that other components, devices, and systems can be integrated into the system 20, such as an endoscope system, a catheterization system, an imaging system, a lighting system, and/or a video camera system, to name a few possibilities. The connection link 50 and the surgical tool 60 are movable toward and away from a surgical site adjacent the spinal column B during a surgical procedure that may utilize one or more retractors, tubes, sleeves, guards, micro-incisions or other components to enhance visualization and clarity.

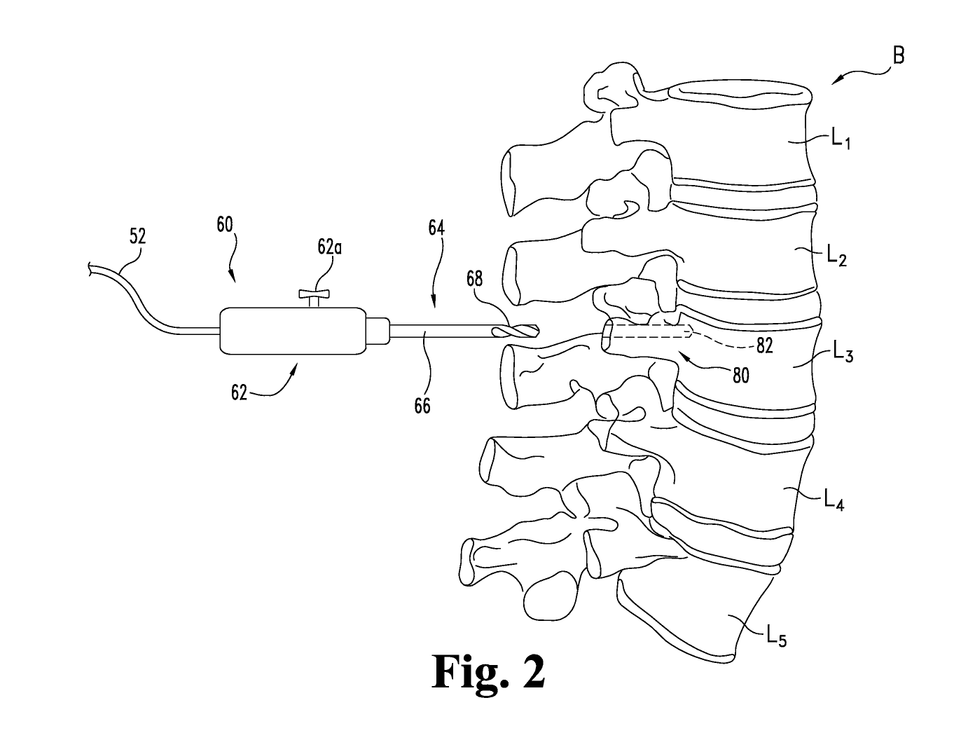

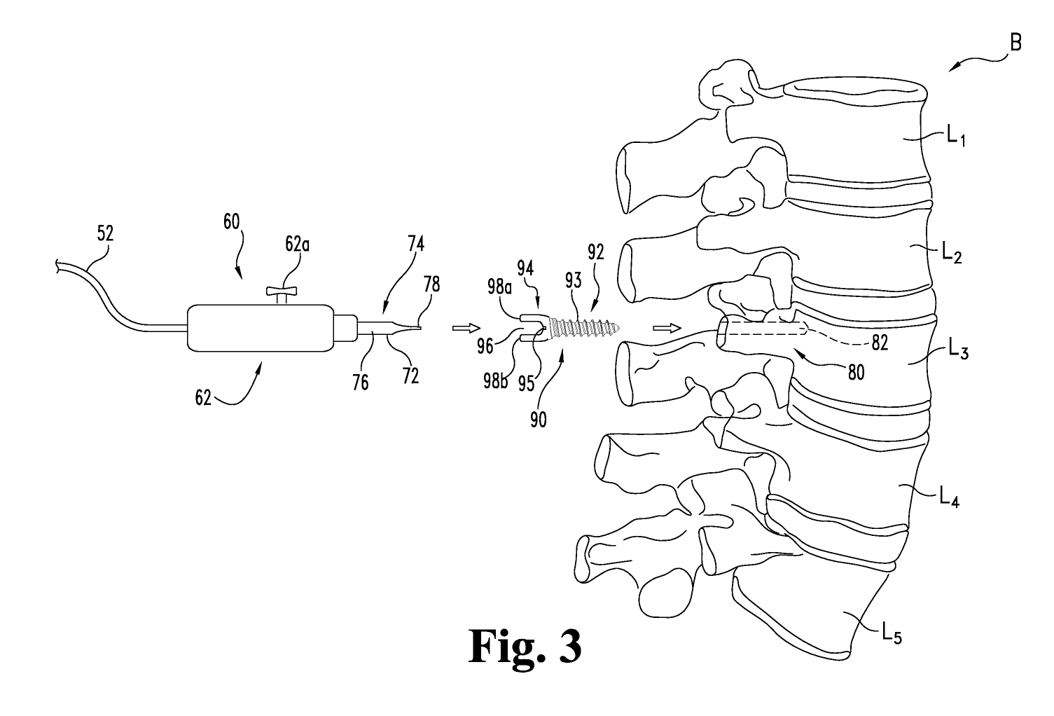

Various embodiments of the surgical tool 60 are illustrated in FIGS. 2 and 3 relative to an implant site 80 in vertebra L₃ of the spinal column B, as viewed laterally from the right side of a patient. It should be understood that throughout FIGS. 1-4, the system 20, the surgical tool 60, and the implants 90 are shown relative to the lumbar region of the spinal column B, including lumbar vertebral bodies L₁-L₅. However, it should be understood that the lumbar region has been shown for illustrative purposes only, and that the systems and methods discussed herein may be applied to any region or vertebral body of the spinal column B.

In FIGS. 1 and 2, the surgical tool 60 generally includes a handle portion 62 and working portion 64 in the form of a bit suitable for use as a drill to cut and remove bone material to form a hole or bore 82 for receipt of a bone anchor. The bit 64 includes a universal connector (not shown) at its proximal end, an elongate shaft 66, and a non-insulated cutting portion 68 at its distal end. The universal connector of the bit 64 may include any suitable configuration for releasable connection with the handle portion 62 of the surgical tool 60. The surgical tool 60 also includes a user control 62a located on the handle portion 62 which may be depressed or activated to supply rotary movement to the bit 64 to form the anchor hole or bore 82 at the implant site 80, as shown in dashed lines in FIGS. 2 and 3. As illustrated in FIGS. 2 and 3, the hole or bore 82 is formed in the pedicle region of the vertebral body L₃ adjacent the pedicle wall. However, it should be understood that one skilled in the art would appreciate that the hole or bore 82 may be formed at other locations or regions of the vertebral body, or in other bone structures outside of the spinal column B.

Referring to FIG. 3, illustrated therein is another embodiment of the surgical tool 60, generally including the handle portion 62 and a working portion 74 in the form of a driver including a universal connector (not shown) at its proximal end, an elongate shaft 76, and a non-insulated driver portion 78 at its distal end. As illustrated in FIG. 3, the distal driver portion 78 is configured for engagement with an implant 90, which in the illustrated embodiment is configured as a bone anchor. The bone anchor 90 includes a longitudinal extending shank portion 92 and a head portion 94 attached to the proximal end of the shank portion 92. In the illustrated embodiment, the shank portion 92 includes helical threads 93 extending along its length, and is structured to threadingly engage a passageway or bore formed in one or more bones or bony structures. The threaded shank portion 92 may also be provided with cutting flutes or other structural features configured to provide the bone anchor 90 with self-tapping and/or self-drilling capabilities. The shank portion 92 may also be cannulated to receive a guidewire to facilitate placement of the bone anchor 90 at the surgical site, and may further include fenestrations or other openings for placement of bone growth material adjacent bone tissue at and around the shank portion 92. Other embodiments of the bone anchor 90 may include alternative configurations of the shank portion 92 for engaging vertebral bone including, for example, non-threaded configurations.

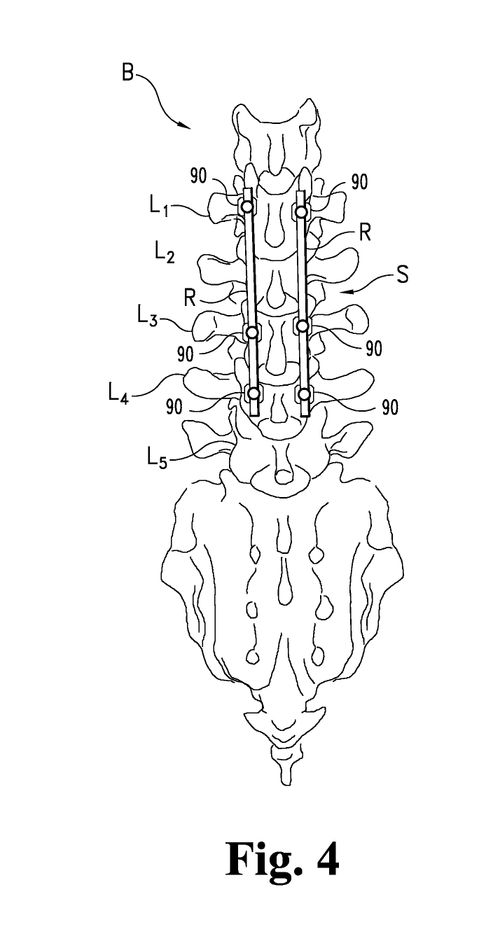

The head portion 94 includes a tool engagement feature 95 and various other features including, for example, a U-shaped receiving channel 96 formed between a pair of arms 98a, 98b. In the illustrated embodiment, the tool engagement feature 95 is configured as a shaped recess or slot sized and shaped for receipt and mating engagement with the distal tip of the driver portion 78 of the surgical tool 60. It should be appreciated that when the driver portion 78 is engaged with the head portion 94, a conductive electrical connection is formed between the bone anchor 90 (including the shank portion 92) and the surgical tool. Although a particular embodiment of the tool engagement feature 95 has been illustrated in FIG. 3, it should be understood that other types and configurations of tool engagement features are also contemplated, including external (i.e., non-recessed) configurations, and other shapes including Phillips, square, hex, star and Torx® shapes. The arms 98a, 98b of the head portion 94 can be internally and/or externally threaded, or may include other features adapted to engage a set screw, a nut, a cap or other clamping devices configured for securing an elongate stabilization element or spinal rod R (FIG. 4) within the channel 96 of the head portion 94. It should be understood that other configurations of the head portion 94 are also contemplated, including head configurations including a proximally extending post that may be provided with either a smooth or threaded outer surface, a rounded or flat head, or any other suitable head configuration that would occur to those skilled in the art.

When the driver portion 78 of the surgical tool 60 is engaged with the head portion 94 of the bone anchor 90, the surgical tool 60 is operable to supply a rotary force to the bone anchor 90 to thread the shank portion 92 into the hole or bore 82 at the surgical site 80. In the illustrated embodiment, the bone anchor 90 is configured as a fixed angle bone screw wherein the shank portion 92 and the head portion 94 are formed as a unitary, single-piece structure. However, it should be understood that the bone anchor 90 may be configured as a multi-axial, poly-axial, uni-axial, multi-planar or uni-planar bone screw wherein the shank portion 92 and the head portion 94 are movable relative to one another in one or more directions or along one or more planes. Furthermore, in one embodiment, the bone anchor 90 is formed of a metallic material such as medical grade stainless steel. However, in other embodiments, the bone anchor 90 may be formed of other metallic materials including titanium, a titanium alloy or other metallic alloys, and/or an electrically conductive nonmetallic material.

As shown in FIGS. 2 and 3, the surgical tool 60 is electrically coupled with the link 52 of the nerve monitoring system 30. Referring to FIG. 2, the nerve monitoring system 30 is operable to detect impingement or interference between the distal cutting portion 68 of the bit 64 and a neural element during formation of the hole 82 in the vertebral body, thereby indicating an exposure, encroachment or close proximity of a neural element adjacent the hole 82. Referring to FIG. 3, the nerve monitoring system 30 is operable to detect impingement, interference, encroachment or close proximity between the shank portion 92 of the bone anchor 90 and a neural element during and after insertion of the shank portion 92 into the hole 82. In some embodiments, upon detection of interference with or exposure to a neural element, the nerve monitoring system 30 may terminate the power supply to the surgical tool 60 to stop movement of the bit portion 64 or the driver portion 64 to avoid potential damage to the neural element. Further details and information regarding neural integrity monitoring and detection systems and devices are set forth in U.S. Pat. No. 5,474,558 to Neubardt, U.S. Patent Publication No. 2006/0173374 to Neubardt et al., U.S. Patent Publication No. 2006/0178593 to Neubardt et al., U.S. Patent Publication No. 2006/0178594 to Neubardt et al., U.S. Patent Publication No. 2006/0173521 to Pond et al., and U.S. Patent Publication No. 2008/0269634 to Young, the contents of each hereby incorporated herein by reference in its entirety.

In one embodiment, during operation of the system 20, the nerve monitoring system 30 supplies the surgical tool 60 with an electrical signal that is used to locate neural elements in contact with or proximate to the distal cutting portion 68 of the bit 64 and/or the shank portion 92 of the bone anchor 90. An electrical lead is positioned in electrical communication with the proximal portion of the bit 64 or the proximal portion of the drive 74, with the lead extending through the handle portion 62 of the surgical tool 60 to the nerve monitoring system 30 for coupling with a source of electrical current, either separately from or part of the connection link 50. In FIG. 2, an electrical signal or current is delivered to the distal cutting portion 68 via the shaft 66 to provide monitoring and detection of neural elements. The distal cutting portion 68 carries an electrical signal that provides an indication of the proximity of neural elements in or adjacent to the bone tissue relative to the distal cutting portion 68 during formation of the hole 82. In FIG. 3, the electrical signal or current is delivered to the distal driver portion 78 via the shaft 76, through the head portion 94 of the bone anchor 90 and on to the shank portion 92 to provides an indication of the proximity of neural elements in the bone tissue relative to the shank portion 92 during and after implantation of the shank portion 92 into the hole 82.

In another embodiment, the electric signal provides electrical stimulation to the tissue surrounding the hole 82, and the patient's response to the nerve stimulation is monitored to determine whether a neural element threshold level has been reached. The threshold level can correspond to, for example, an indication of the presence of a neural element and/or its proximity relative to hole 82 and are the shank portion 92 of the bone anchor. In another embodiment, when the source of the electrical current (either the distal cutting portion 68 of the bit 64 or the shank portion 92 of the bone anchor 90) is positioned near or proximate a neural element, the presence of the neural element creates an electrical current path for conduction of an electrical signal. The current path provides an indication to the nerve monitoring system 30 corresponding to the presence of the neural element, and corrective action can then be taken by the surgeon based on this indication. In other words, detection of the neural element threshold occurs as a function of the electrical signal at either the distal cutting portion 68 of the bit 64 or the shank portion 92 of the bone anchor 90, thereby inducing a reaction in the patient or a particular reading of the threshold level.

In some embodiments, certain components of the system 20 comprise an electrically conductive material surrounded by an insulative member or coating to prevent shunting of an electrical current or signal delivered therethrough to adjacent tissue or devices. For example, the link 52 and/or the handle portion 62 of the tool 60 may include an electrical pathway surrounded by an insulative material. Furthermore, the universal connectors (not shown) located at the distal end of the handle portion 62 and/or the shafts 66, 76 of the working portions 64, 74 may also be insulated to prevent electrical shunting. However, the distal cutting portion 68 and the distal driver portion 78 are not insulated. Specifically, the distal cutting portion 68 is not insulated so as to conductively expose the distal cutting portion 68 to adjacent bone tissue to allow conductive transfer of an electrical signal from the adjacent bone tissue to the distal cutting portion 68 for monitoring and detection of nerve proximity. The distal driver portion 78 is likewise not insulated so as to conductively couple the distal driver portion 78 to the head portion 94 of the bone anchor to allow conductive transfer of an electrical signal from the adjacent bone tissue to the shank portion 92 of the bone anchor for monitoring and detection of nerve proximity. In some embodiments, the entire bone anchor 90 is not insulated. However, in other embodiments, portions of the bone anchor 90, such as regions of the head portion 94 and select regions of the shank portion 92, may be insulated to prevent shunting and interference from surrounding tissues or instruments.

Referring to FIG. 4, shown therein is a posterior view a portion of the spinal column B of a patient including lumbar vertebra L₁-L₅, with a spinal stabilization or fixation system S attached to multiple levels of the lumbar region of the spinal column B via a plurality of the bone anchors 90. However, it should be understood that the devices, systems and methods discussed herein are also applicable to other regions of the spinal column B, including the cervical, thoracic and sacral regions of the spinal column B. As shown in FIG. 4, a number of the bone anchors 90 have been anchored to multiple vertebrae of the spinal column B in accordance with the systems and methods described above. Additionally, elongate stabilization elements R are engaged to the bone anchors 90 by appropriate means, the details of which would be apparent to those skilled in the art. The spinal stabilization or fixation system S may be used to address numerous deformities or abnormalities associated with spinal column including, for example, treatment of degenerative spondylolisthesis, fractures, dislocations, scoliosis, kyphosis, spinal tumors, and/or a failed fusion attempt, just to name a few examples. In the illustrated embodiment of the spinal stabilization system S, the bone anchors 90 are configured as pedicle bone screws and the elongate stabilization elements R are configured as spinal rods. However, other types and configurations of the bone anchors 90 and the elongate stabilization elements R are also contemplated.

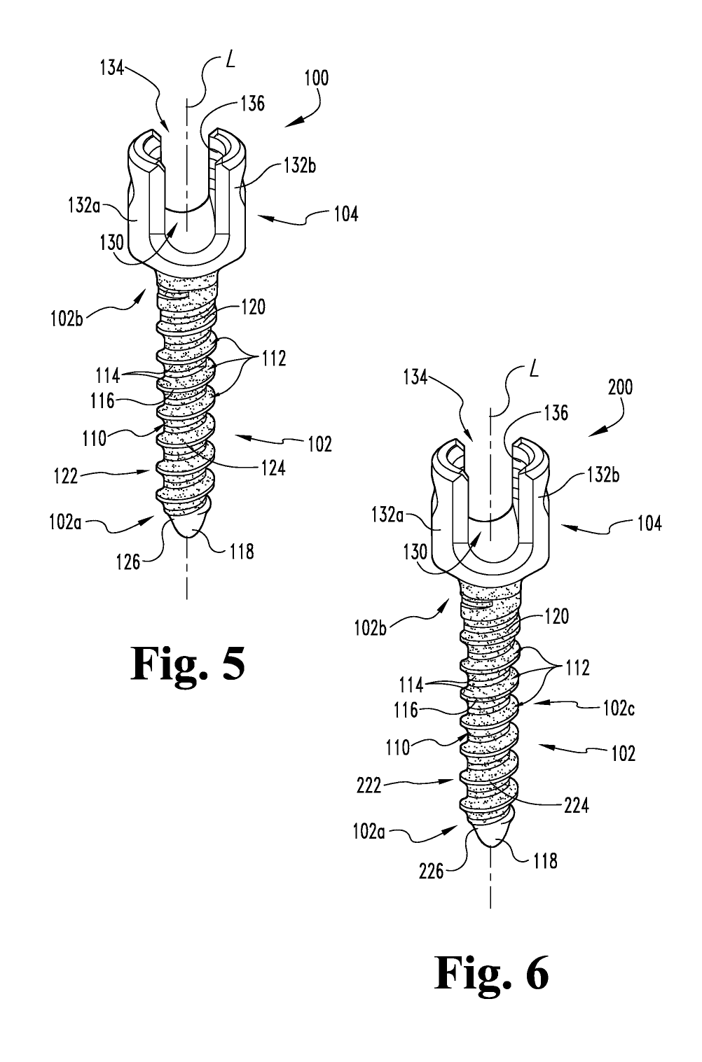

Referring to FIG. 5, shown therein is a bone anchor 100 according to one form of the present invention compatible for use with neural integrity monitoring systems and procedures. The bone anchor 100 extends along a longitudinal axis L and generally includes a bone engaging portion 102 configured for anchoring in or to bone, and an implant engaging portion or head 104 configured for engaging an implant or another device. The bone engaging portion 102 includes a distal end region 102a and a proximal end region 102b, with the implant engaging portion 104 extending from the proximal end region 102b. In the illustrated embodiment, the bone anchor 100 is a bone screw, with the bone engaging portion 102 comprising an at least partially threaded shank configured for threading engagement within bone, and the implant engaging portion 104 comprising a screw head adapted for operative engagement with an implant such as, for example, the elongate spinal rod R illustrated in FIG. 4 and described above. Further details regarding the bone engaging portion 102 and the implant engaging portion 104 of the bone anchor 100 will be discussed below.

Although the bone anchor 100 has been illustrated as having a particular configuration of the bone engaging portion 102 and a particular configuration of the implant engaging portion 104, other configurations are also contemplated. For example, although the illustrated embodiment of the bone engaging portion 102 is configured as a threaded shank, the bone engaging portion 102 may alternatively be configured as a fusion device or hollow cage, a bolt, a pin or nail having a non-threaded configuration, a laminar hook, a clamp, a staple, and other types of bone engaging structures capable of being anchored in or to bone. Additionally, although the illustrated embodiment of the implant engaging portion 104 is configured as a U-shaped head sized and shaped for engagement with an elongate spinal rod R, in other embodiments, the implant engaging portion 104 may be configured for engagement with other types of stabilization elements or devices such as, for example, tethers, cables, wires, bands, sutures, plates, connectors, intervertebral implants, intravertebral implants, or other types of spinal implants or load carrying/stabilization devices know to those skilled in the art. Additionally, the stabilization elements may be solid or hollow, rigid, flexible or partially flexible, circular or non-circular, and may have a homogenous or heterogeneous material composition.

In one embodiment, the bone engaging portion 102 of the bone anchor 100 is configured to be anchored within bone, and the implant engaging portion 104 is positioned outside of the bone. However, in other embodiments, the bone engaging portion 102 may be at least partially engaged to an exterior portion of the bone, such as is the case, for example, with bone anchors having a hook-type or clamp-type configuration. In still other embodiments, the implant engaging portion 104 may be positioned partially within or entirely within the bone. Additionally, the bone anchor 100 may be used to engage an orthopedic implant to bone, and more specifically a spinal implant, to vertebral bone. However, it should be understood that the bone anchor 100 may be used in association with other types of implants outside of the orthopedic field or the spinal field.

In the illustrated embodiment, the bone engaging portion 102 includes an elongate shank 110 having bone anchoring elements 112 extending therefrom which are configured to engage bone, particularly vertebral bone, and more particularly cancellous vertebral bone. In one embodiment of the invention, the bone anchoring elements 112 are configured as one or more threads extending helically about the shank 110 and along the longitudinal axis L. However, it should be understood that other types and configuration of bone anchoring elements are also contemplated as falling with the scope of the present invention including, for example, multiple thread-like elements which are formed by circumferentially, radially or axially interrupting a single thread, various types of raised projections extending about the outer periphery of the elongate shank 110 along a generally helical path, various types of grooves or recesses extending about the outer periphery of the elongate shaft 110 along a generally helical path, ratchet elements that allow for relatively uninhibited insertion into bone in a first direction but which resist movement in an opposite second direction, spikes, annular or circular ridges, teeth, surface roughening, or any other type bone anchoring element that would occur to those skilled in the art.

In the illustrated embodiment, the bone engaging portion 102 includes a single lead thread 112 having a uniform pitch, a substantially uniform outer thread diameter, and a substantially uniform inner thread root diameter. However, in other embodiments, the bone engaging portion 102 may include a multi-lead thread, a variable thread pitch, a tapered outer thread diameter and/or a tapered inner thread root diameter. Additionally, the thread 112 includes upper and lower thread flank surfaces 114 extending from the shank 110 to an outer thread crest surface or edge 116. In the illustrated embodiment, the upper and lower flank surfaces 114 are relatively flat and obliquely angled relative to the longitudinal axis L, and the outer crest surface 116 is relatively flat and arranged generally parallel with the longitudinal axis L. However, other types and configurations of threads are also contemplated as falling within the scope of the present invention, including threads having a pointed or rounded outer crest surface and/or thread configurations wherein one or both of the upper and lower flank surfaces are rounded or extend in a direction substantially normal to the longitudinal axis L.

Additionally, in the illustrated embodiment of the bone anchor 100, the distal tip portion 118 is tapered and defines a rounded tip. However, in other embodiments, the distal tip portion 118 may define a pointed tip to facilitate penetration into bone, may define a blunt or substantially flat end surface, or may be provided with cutting elements, such as cutting teeth, to facilitate entry into bone. In further embodiments, the distal end region 102a of the bone engaging portion 102 may be provided with one or more cutting edges or flutes (not shown) to provide the bone anchor 100 with self-cutting or self-tapping capabilities. In still other embodiments, the bone anchor 100 may be provided with an axial passage or cannulation opening (not shown) extending either partially or entirely through the bone engaging portion 102, and may be further provided with transverse passages that communicate with the axial passage to define fenestration openings. The cannulation opening may be sized to receive an elongate member, such as a guide wire, to guide the bone anchor into a desired location adjacent the surgical site, and/or to guide other components into engagement with the bone anchor. Additionally, the cannulation and fenestration openings may be used to deliver material such as, for example, bone cement, through the bone engaging portion 102 and into areas of the bone axially or laterally adjacent the distal end region 102a or laterally adjacent other portions of the bone engaging portion 102.

In one embodiment of the invention, the implant engaging portion 104 is integral with the bone engaging portion 102 to define a unitary bone anchor 100. More specifically, the threaded shank 102 and the screw head 104 are formed as a unitary, single-piece structure. However, in other embodiments, the threaded shank 102 and the screw head 104 may be formed as separate components and coupled to one another by any suitable attachment or connection technique, such as, for example, by welding, bonding, fusing, fastening, pinning, or by any other technique or process that would occur to one of ordinary skill in the art. Additionally, the threaded shank 102 and the screw head 104 may be formed as separate components that are subsequently coupled or assembled together by any suitable attachment or connection technique. In other embodiments, the threaded shank 102 and the screw head 104 may be formed as separate components that are subsequently coupled or assembled together in a movable manner to allow for relative movement between the threaded shank 102 and the screw head 104 along one or more axes and along one or more planes to provide the bone screw 100 with multi-axial, uni-axial, multi-planar or uni-planar characteristics.

In the illustrated embodiment of the bone anchor 100, the implant engaging portion 104 is configured for engagement with the elongate spinal rod R (FIG. 4). In one specific embodiment, the screw head 104 defines a U-shaped passage 130 sized to receive the spinal rod R, with a fastener or setscrew (not shown) extending into the passage 130 and into engagement with the spinal rod to capture and secure the spinal rod to the screw head 104. In another specific embodiment, the screw head 104 includes a pair of spaced apart arms 132a, 132b defining an open end 134 which provide the passage 130 with a top-loading configuration, with the fastener or setscrew engaged with internal threads 136 formed along the spaced apart arms 132a, 132b. Further details regarding bone screws having configurations similar to the configuration of the bone anchor 100 are illustrated and described, for example, in U.S. Pat. No. 6,783,527 to Drewry et al., the contents of which are incorporated herein by reference.

In other embodiments of the bone anchor 100, the implant engaging portion 104 may be configured as an unthreaded stem portion or shaft, with the spinal rod coupled to the stem portion via a connector or coupling mechanism that includes a connector body defining a first passage for receiving the stem portion, and a second passage for receiving the spinal rod. One or more fasteners or set screws may be threaded through corresponding openings in the connector body to secure the connector body to the stem portion of the bone screw and to the spinal rod. Further details regarding bone screw configurations and a connector or coupling mechanisms suitable for use in association with the present invention are illustrated and described, for example, in U.S. Pat. No. 5,663,263 to Simonson and U.S. Pat. No. 5,947,957 to Barker, the contents of each patent reference hereby incorporated herein by reference. It should be understood that the implant engaging portion 104 of the bone anchor 100 may be provided with other configurations suitable for engaging an implant, the details of which would be apparent to those skilled in the art.

The bone anchor 100 may be formed of various biocompatible materials suitable for implantation within the body and which are capable of conducting an electrical current or signal to render the bone anchor suitable for use with neural integrity or nerve monitoring systems, the details of which have been set forth above. In one embodiment, the bone anchor 100 is formed at least partially from a metallic material such as, for example, a medical grade stainless steel. However, other metallic materials are also contemplated, including titanium, titanium alloys, stainless steel alloys, chrome-cobalt alloys or shape-memory alloys. Additionally, the bone anchor 100 may be formed of any non-metallic material suitable for implantation within the body and which is capable of conducting an electrical current or signal to render the bone anchor suitable for use with neural integrity or nerve monitoring systems.

In order to facilitate bone growth onto/into the bone engaging portion 102 of the bone anchor 100 when anchored in or to bone to thereby increase the purchase strength of the connection with bone, select regions of the bone engaging portion 102 are at least partially coated with a bone growth promoting material 120. In one embodiment, the coating of bone growth promoting material 120 comprises a calcium phosphate material such as, for example, hydroxyapatite. However, other types of bone growth promoting materials are also contemplated for use in association with the present invention, the likes of which would be apparent to those skilled in the art. As would be appreciated by those skilled in the art, applying a coating of bone growth promoting material 120 to the bone engaging portion 102 can act as an insulator and/or interfere with or significantly weaken the conductive electrical path between the bone engaging portion 102 of the bone anchor 100 and the adjacent tissue, the likes of which are necessary for proper operation of the system 20 illustrated in FIGS. 1-3 and described above. As discussed above, a conductive electrical current path between the bone engaging portion of the bone anchor and adjacent neural elements or nerves is needed to provide an indication to the nerve monitoring system 30 corresponding to the presence of a neural element so that corrective action can then be taken by the surgeon based on this indication.

In order to maintain a conductive electrical current path between the bone anchor 100 and the adjacent neural elements or nerves, one or more portions of the bone engaging portion 102 are provided with conductive surface regions or areas 122 that are contiguous with and positioned adjacent to the insulated or coated regions including the bone growth promoting material 120. The conductive surface areas or regions 122 exhibit a higher electrical conductance value relative to the insulated regions of the bone engaging portion 102 that are coated with the bone growth promoting material 120. In other words, the conductive surface areas or regions 122 of the bone engaging portion 102 exhibit less electrical resistance relative to the coating of bone growth promoting material 120 to establish a conductive pathway between the bone anchor 100 and the adjacent neural elements or nerves to facilitate operation of the nerve monitoring system 30 illustrated in FIGS. 1-3 and described in detail above.

In one form of the present invention, the conductive surface areas 122 constitute regions of the bone engaging portion 102 that do not include the bone growth promoting coating 120. In one embodiment, the conductive surface areas 122 constitute exposed metallic surfaces of the bone engaging portion 102. However, in other embodiments, the conductive surface areas 122 may constitute regions of the bone engaging portion 102 that are provided with a reduced thickness of the bone growth promoting coating 120 sufficient to maintain a conductive electrical current path between the bone anchor 100 and adjacent neural elements or nerves. In still other embodiments, the conductive surface areas 122 may constitute regions of the bone engaging portion 102 that are provided with a conductive material or coating that maintains a conductive electrical current path between the bone anchor 100 and adjacent neural elements or nerves.

In the illustrated embodiment, the outer thread crest surface or edge 116 of the helical thread 112 is provided with a conductive surface 124 extending from the distal end region 102a to the proximal end region 102b of the bone engaging portion 102. Additionally, the distal tip portion 118 may also be provided with a conductive surface 126. As would be appreciated by those skilled in the art, the outer thread crest surface or edge 116 of the helical thread 112 and the distal tip portion 118 are typically the first portions of the bone anchor 100 that make contact with or are positioned closest in proximity to neural elements or nerves when the bone anchor 100 is driven into bone. Accordingly, a conductive electrical current path between the bone anchor 100 and the adjacent neural elements or nerves may be maintained via providing the conductive surface 124 along the outer thread crest surface 116 of the helical thread 112 and the conductive surface 126 along the distal tip portion 118, thereby facilitating proper operation of the neural integrity or nerve monitoring system 30 to accurately monitor and detect neural elements during or subsequent to anchoring of the bone engaging portion 102 in bone.

As should be appreciated, providing the bone engaging portion 102 with the conductive surfaces 122 can be achieved by a variety of techniques and procedures. For example, in one embodiment, the bone engaging portion 102 is entirely coated with the bone growth promoting material 120, followed by removal of the bone growth promoting material 120 from the outer thread crest surface 116 along one or more regions of the helical thread 112 and/or along the distal tip portion 118. Removal of the bone growth promoting material 120 from the outer thread crest surface 116 can be accomplished by a machining or finishing operation such as, for example, grinding, scraping, polishing or other machining or finishing operations known to those skilled in the art, or by a chemical operation such as, for example, etching or other chemically induced removal operations known to those skilled in the art. In another embodiment, the outer region of the helical thread 112 may be sharpened to remove the bone growth promoting material 120 and to provide the helical thread 112 with a sharpened tip to facilitate cutting/threading into bone.

In a further embodiment, providing the bone engaging portion 102 with the conductive surfaces 122 can be achieved by a masking operation wherein the outer thread crest surface 116 along one or more regions of the helical thread 112 are masked or covered prior to coating the exposed regions of the bone engaging portion 102 (including the shank 110 and the upper and lower flank surfaces 114) with the bone growth promoting material 120. The masking agent may include tape, a liquid or gel, a hard fixture such as a plastic tube, or any other masking agent or device known to those skilled in the art. After the coating of the bone growth promoting material 120 is applied to the exposed regions of the bone engaging portion 102, the masking is removed, thereby exposing a non-coated conductive surface 124 along the outer thread crest surface 116 or along other portions of the helical thread 112 or shank 110. In some instances, the masking agent need not be removed, particularly in cases where the masking agent has good electrically conductive properties.

In another embodiment, the coating of the bone growth promoting material 120 may be applied to select regions of the helical thread 112 (such as the shank 110 and the upper and lower flank surfaces 114) while avoiding application of the bone growth promoting material 120 coating to other regions of the helical thread (such as the outer thread crest surface 116). This selective application operation may be accomplished by a robotics system or an automated process for improved accuracy. In a further embodiment, a masking operation may be utilized prior to surface treatment or preparation of the bone engaging portion 102 to more readily accept the coating of the bone growth promoting material 120. For example, the outer thread crest surface 116 along one or more regions of the helical thread 112 may be masked or covered by a masking agent or device prior to treatment/preparation of the exposed surfaces such as, for example, by a media blast operation. The masking is later removed followed by coating of the bone engaging portion 102 with the bone growth promoting material 120. However, the previously masked areas that were not exposed to the surface treatment/preparation process can be more easily cleaned to remove the bone growth promoting material 120 from the masked regions to thereby provide the conductive surface areas.

It should be appreciated that other techniques and procedures for providing the bone engaging portion 102 of the bone anchor 100 with the conductive surface areas 122 are also contemplated, the likes of which would be apparent to those skilled in the art.

Referring to FIG. 6, shown therein is a bone anchor 200 according to another form of the present invention. The bone anchor 200 is configured similar to the bone anchor 100 illustrated in FIG. 5 and described above. Accordingly, like elements and features are indicated using the same reference numbers. However, the bone anchor 200 includes a different configuration of conductive surface areas 222. Specifically, the outer thread crest surface or edge 116 of the helical thread 112 is provided with a conductive surface 224 extending from the distal end region 102a to a central or mid-region 102c of the bone engaging portion 102. Additionally, the distal tip portion 118 is also provided with a conductive surface 226. However, the proximal portion of the bone engaging portion 102, including the outer thread crest surface or edge 116 of the helical thread 112, is coated with the bone growth promoting material 120 from the mid-region 102c to the proximal end region 102b of the bone engaging portion 102.

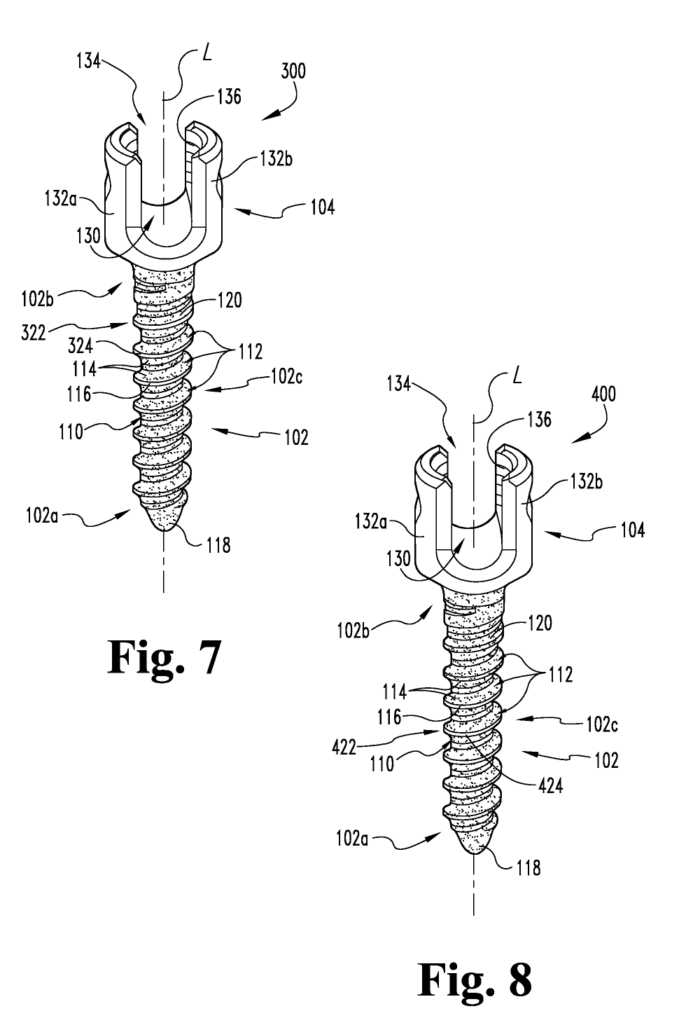

Referring to FIG. 7, shown therein is a bone anchor 300 according to another form of the present invention. The bone anchor 300 is configured similar to the bone anchor 100 illustrated in FIG. 5 and described above. Accordingly, like elements and features are indicated using the same reference numbers. However, the bone anchor 300 includes a different configuration of conductive surface areas 322. Specifically, the outer thread crest surface or edge 116 of the helical thread 112 is provided with a conductive surface 324 extending from the proximal end region 102b to a central or mid-region 102c of the bone engaging portion 102. However, the distal portion of the bone engaging portion 102, including the outer thread crest surface or edge 116 of the helical thread 112, is coated with the bone growth promoting material 120 from the mid-region 102c to the distal end region 102a of the bone engaging portion 102. Additionally, the distal tip portion 118 is also coated with the bone growth promoting material 120.

Referring to FIG. 8, shown therein is a bone anchor 400 according to another form of the present invention. The bone anchor 400 is configured similar to the bone anchor 100 illustrated in FIG. 5 and described above. Accordingly, like elements and features are indicated using the same reference numbers. However, the bone anchor 400 includes a different configuration of conductive surface areas 422. Specifically, the outer thread crest surface or edge 116 of the helical thread 112 is provided with a conductive surface 424 extending along the central or mid-region 102c of the bone engaging portion 102. However, the proximal and distal portions of the bone engaging portion 102, including the outer thread crest surface or edge 116 of the helical thread 112, are coated with the bone growth promoting material 120. Additionally, the distal tip portion 118 is also coated with the bone growth promoting material 120.

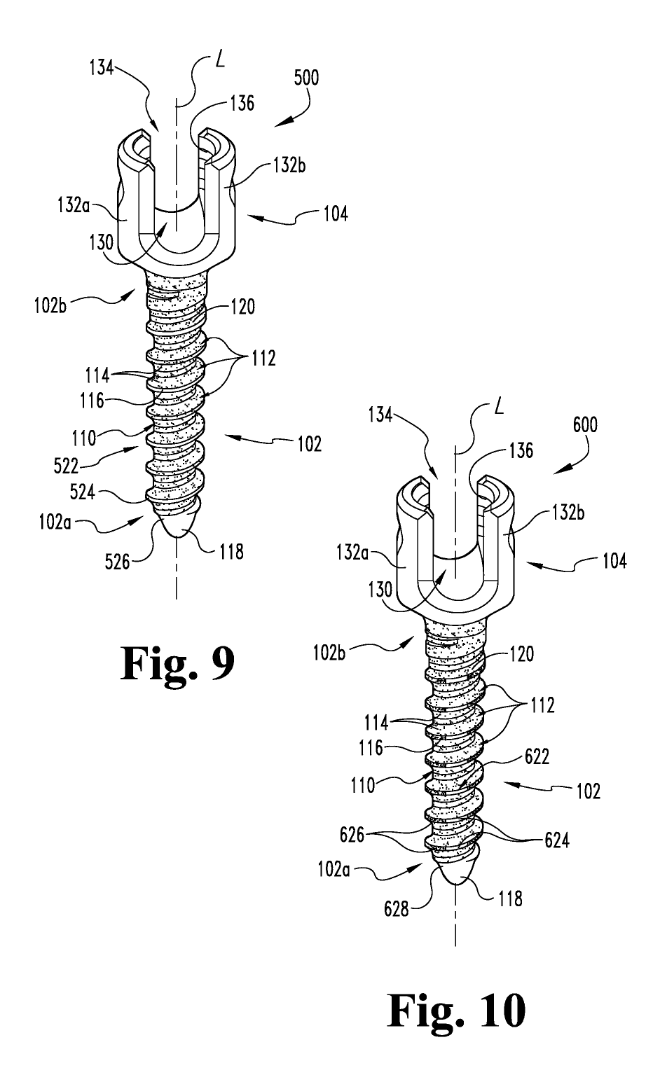

Referring to FIG. 9, shown therein is a bone anchor 500 according to another form of the present invention. The bone anchor 500 is configured similar to the bone anchor 100 illustrated in FIG. 5 and described above. Accordingly, like elements and features are indicated using the same reference numbers. However, the bone anchor 500 includes a different configuration of conductive surface areas 522. Specifically, the outer thread crest surface or edge 116 of the helical thread 112 is provided with a conductive surface 524 extending along alternating turns of the helical thread 112. In other words, the outer thread crest surface 116 of every other turn of the helical thread 112 is provided with a conductive surface area 524, with the outer thread crest surface 116 of the intervening turns coated with the bone growth promoting material 120. Additionally, the distal tip portion 118 is also provided with a conductive surface 526.

Referring to FIG. 10, shown therein is a bone anchor 600 according to another form of the present invention. The bone anchor 600 is configured similar to the bone anchor 100 illustrated in FIG. 5 and described above. Accordingly, like elements and features are indicated using the same reference numbers. However, the bone anchor 600 includes a different configuration of conductive surface areas 622. Specifically, the outer thread crest surface or edge 116 of the helical thread 112 is provided with intermittent conductive surface areas 624 that are generally aligned in columns along the longitudinal axis L. In other words, the outer thread crest surface 116 of the helical thread 112 is provided with alternating conductive surface areas 624 and coated surface areas 626 that are coated with the bone growth promoting material 120, with the conductive surface areas 624 of each adjacent thread turn generally aligned in columns along the longitudinal axis L. Additionally, the distal tip portion 118 is also provided with a conductive surface 628. In an alternative embodiment, the intermittent conductive surface areas 624 of the thread turns can be circumferentially offset from the intermittent conductive surface areas 624 of the adjacent thread turns (i.e., the intermittent conductive surface areas 624 are not aligned in columns along the longitudinal axis L).

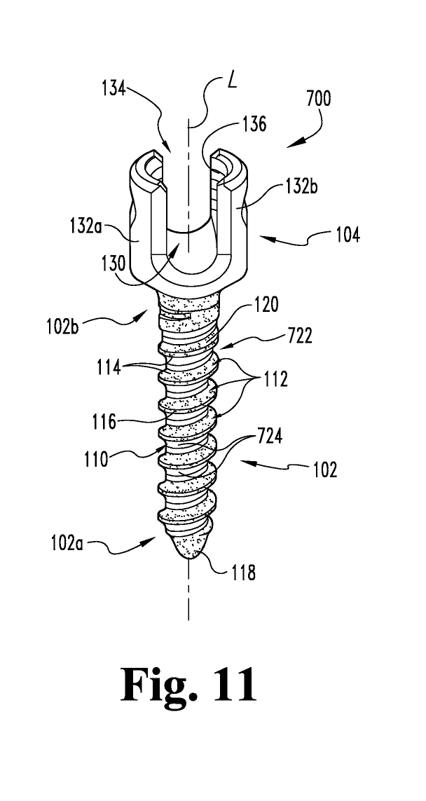

Referring to FIG. 11, shown therein is a bone anchor 700 according to another form of the present invention. The bone anchor 700 is configured similar to the bone anchor 100 illustrated in FIG. 5 and described above. Accordingly, like elements and features are indicated using the same reference numbers. However, the bone anchor 700 includes a different configuration of conductive surface areas 722. Specifically, the shank region 110 of the bone engaging portion 102 between the turns of the helical thread 112 is provided with a conductive surface area 724 extending from the distal end region 102a to the proximal end region 102b. However, the helical thread 112, including the upper and lower flank surfaces 114 and the outer crest surface 116, is coated with the bone growth promoting material 120. Additionally, the distal tip portion 118 is also coated with the bone growth promoting material 120.

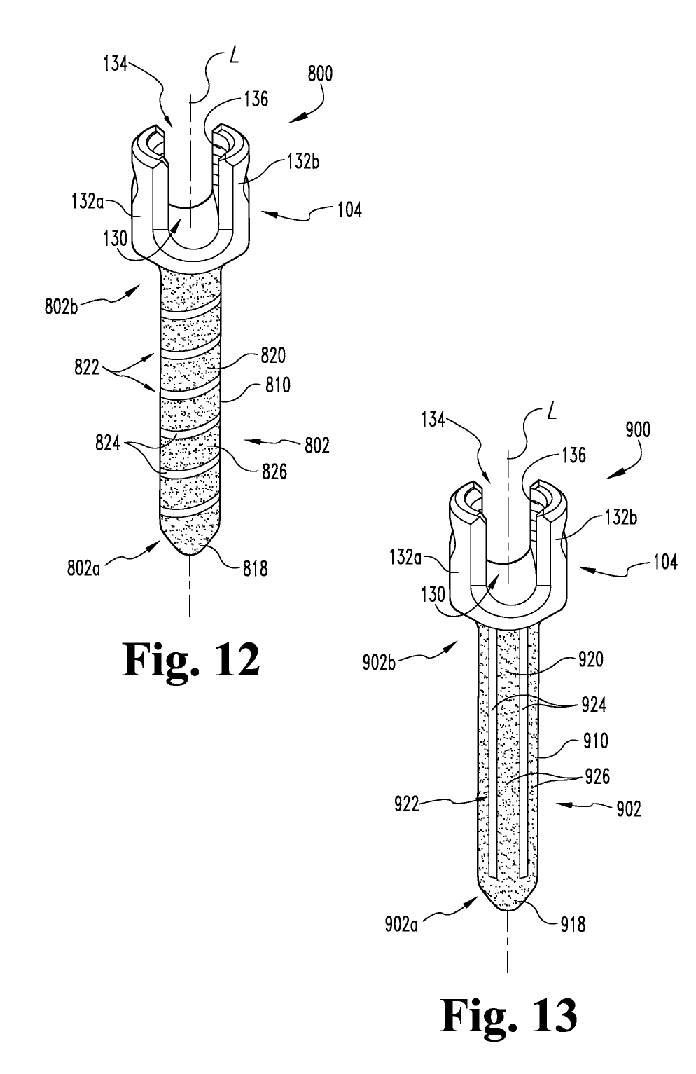

Referring to FIG. 12, shown therein is a bone anchor 800 according to another form of the present invention. The bone anchor 800 is configured, in some respects, similar to the bone anchor 100 illustrated in FIG. 5 and described above. Accordingly, like elements and features are indicated using the same reference numbers. However, unlike the bone anchor 100 which includes a threaded bone engaging portion 102, the bone anchor 800 includes a bone engaging portion 802 including a non-threaded shank 810 extending from the distal end region 802a to the proximal end region 802b. Additionally, the bone engaging portion 802 is partially coated with a bone growth promoting material 820 like that of the bone growth promoting material 120 described above. However, portions of the bone engaging portion 802 are provided with conductive surface areas 822. In the illustrated embodiment, the conductive surface areas 822 are configured as conductive surface rings 824 extending annularly about the non-threaded shank 810 and offset from one another along the longitudinal axis L. In other words, the non-threaded shank 810 is provided with alternating rings of conductive surface areas 824 and coated surface areas 826 that are coated with the bone growth promoting material 820. Additionally, the distal tip portion 818 of the non-threaded shank 810 is also coated with the bone growth promoting material 820.

Referring to FIG. 13, shown therein is a bone anchor 900 according to another form of the present invention. The bone anchor 900 is configured, in some respects, similar to the bone anchor 100 illustrated in FIG. 5 and described above. Accordingly, like elements and features are indicated using the same reference numbers. However, unlike the bone anchor 100 which includes a threaded bone engaging portion 102, the bone anchor 900 includes a bone engaging portion 902 including a non-threaded shank 910 extending from the distal end region 902a to the proximal end region 902b. Additionally, the bone engaging portion 902 is partially coated with a bone growth promoting material 920 like that of the bone growth promoting material 120 described above. However, portions of the bone engaging portion 902 are provided with conductive surface areas 922. In the illustrated embodiment, the conductive surface areas 922 are configured as a number of conductive surface columns 924 extending axially along the length of the non-threaded shank 910 along the longitudinal axis L. In other words, the non-threaded shank 910 is provided with alternating columns of conductive surface areas 924 and coated surface areas 926 that are coated with the bone growth promoting material 920. Additionally, the distal tip portion 918 of the non-threaded shank 910 is also coated with the bone growth promoting material 920.

It should be understood that any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. A bone anchor compatible for use with a neural integrity monitoring system, the bone anchor comprising:
a bone engaging portion configured for anchoring to bone, said bone engaging portion including at least one insulated region coated with a bone growth promoting material, said bone engaging portion including at least one conductive region contiguous with said insulated region and having reduced electrical resistance relative to said insulated region.

2. The bone anchor of claim 1, wherein said bone growth promoting material comprises a calcium phosphate material.

3. The bone anchor of claim 2, wherein said calcium phosphate material comprises hydroxyapatite.

4. The bone anchor of claim 1, wherein said conductive region extends annularly about a longitudinal axis of said bone engaging portion.

5. The bone anchor of claim 4, wherein said conductive region extends helically about said longitudinal axis of said bone engaging portion.

6. The bone anchor of claim 1, wherein said conductive region extends axially along a longitudinal axis of said bone engaging portion.

7. The bone anchor of claim 1, wherein said bone engaging portion comprises a shank including at least one thread lead; and
wherein said conductive region comprises at least a portion of an outer thread crest of said thread lead.

8. The bone anchor of claim 7, wherein said conductive region comprises an exposed metallic surface extending along said outer thread crest of said thread lead that is not coated with said bone growth promoting material.

9. The bone anchor of claim 7, wherein said conductive region extends continuously along said outer thread crest of said thread lead from a proximal end region of said bone engaging portion to a distal end region of said bone engaging portion.

10. The bone anchor of claim 7, wherein said conductive region comprises intermittent surface areas of said outer thread crest of said thread lead.

11. The bone anchor of claim 7, wherein upper and lower flank surfaces of said thread lead and surfaces of said shank between adjacent turns of said thread lead are coated with said bone growth promoting material.

12. The bone anchor of claim 1, wherein said bone engaging portion is formed of a metallic material, and wherein said conductive region comprises an exposed metallic surface of said bone engaging portion that is not coated with said bone growth promoting material.

13. The bone anchor of claim 1, wherein said conductive region is coated with a layer of said bone growth promoting material having a reduced thickness relative to said bone growth promoting material of said insulated region.

14. The bone anchor of claim 1, further comprising an implant engaging portion configured for attachment to an implant.

15. The bone anchor of claim 1, wherein said at least one conductive region of said bone engaging portion is positioned between and contiguous with two of said insulated regions of said bone engaging portion.

16. The bone anchor of claim 1, wherein said at least one conductive region comprises an outermost region of said bone engaging portion.

17. The bone anchor of claim 16, wherein said outermost region of said bone engaging portion comprises an outer thread crest of a thread lead extending about said bone engaging portion.

18. The bone anchor of claim 1, wherein said at least one conductive region of said bone engaging portion exhibits a higher electrical conductance value relative to adjacent tissue compared to said coating of bone growth promoting material.

19. A system for monitoring neural elements, comprising:
the bone anchor of claim 1; and
a nerve monitoring system electrically coupled to said bone anchor to provide an electrical signal between said conductive region of said bone engaging portion and an adjacent neural element.

20. A bone anchor compatible for use with a neural integrity monitoring system, the bone anchor comprising:
an implant engaging portion configured for engagement with an implant; and
a bone engaging portion extending from said implant engaging portion and configured for anchoring in bone, said bone engaging portion including a shank and at least one thread lead extending about said shank, said shank and upper and lower flank surfaces of said thread lead coated with a bone growth promoting material and defining an insulted region of said bone engaging portion, an outer thread crest of said thread lead defining a conductive region of said bone engaging portion having reduced electrical resistance relative to said insulated region.

21. The bone anchor of claim 20, wherein said bone growth promoting material comprises a calcium phosphate material.

22. The bone anchor of claim 20, wherein said conductive region extends continuously along said outer thread crest of said thread lead from a proximal end region of said bone engaging portion to a distal end region of said bone engaging portion.

23. The bone anchor of claim 20, wherein said conductive region comprises an exposed metallic surface extending along said outer thread crest of said thread lead that is not coated with said bone growth promoting material.

24. The bone anchor of claim 20, wherein said conductive region of said bone engaging portion is not coated with said bone growth promoting material.

25. A bone anchor compatible for use with a neural integrity monitoring system, the bone anchor comprising:
a head configured for engagement with an implant; and
a threaded shank formed of a metallic material and extending from said head, said threaded shank including at least one thread lead, said threaded shank entirely coated with a bone growth promoting material except for a non-coated outer thread crest of said thread lead, said non-coated outer thread crest defining an exposed metallic surface.

26. The bone anchor of claim 25, wherein said bone growth promoting material comprises a calcium phosphate material.

27. The bone anchor of claim 25, wherein said exposed metallic surface extends continuously along said outer thread crest from a proximal end region of said threaded shank to a distal end region of said threaded shank.
